(19) United States
(12) Patent Application Publication
(10) Pub. No.: US 2026/0151581 A1
(43) Pub. Date: Jun. 4, 2026

(54) System and Method for Real-Time Carbon Dioxide and Pressure Sensing to Verify Placement of Tube in Airway or Esophagus

(71) Applicant: Avent, Inc., Alpharetta, GA (US)

(72) Inventors: James F. Tassitano, Marietta, GA (US); Shawn G. Purnell, Sandy Springs, GA (US); Don J. McMichael, Roswell, GA (US); Daniel J. Rogers, Roswell, GA (US)

(21) Appl. No.: 19/459,421

(22) Filed: Jan. 26, 2026

Related U.S. Application Data
(63) Continuation of application No. 17/007,540, filed on Aug. 31, 2020.

(51) Int. Cl. A61M 16/04; A61M 1/00; A61M 16/00

(52) U.S. Cl. CPC A61M 16/0488; A61M 1/84; A61M 16/0003; A61M 16/0463; A61M 2016/0027; A61M 2016/0033; A61M 2202/0225; A61M 2205/3334; A61M 2205/3561; A61M 2205/52; A61M 2205/583; A61M 2205/82; A61M 2210/0618; A61M 2210/0625; A61M 2210/105

(57) ABSTRACT

A catheter sensor assembly for use in conjunction with electronic catheter guidance systems is provided and includes a catheter and a sensor. The catheter extends in a longitudinal direction and has a proximal end and a distal end that define a lumen therebetween. Further, the catheter is configured for placement within a digestive tract or respiratory tract of a patient. The sensor includes a carbon dioxide sensor, pressure sensor, or both, and can be located in an air sampling chamber connected to the catheter. The sensor can communicate with a processor to deliver carbon dioxide and/or pressure readings to a display device, which can indicate placement of the catheter in the digestive tract or in the respiratory tract. A catheter guidance system and a method for accurately placing a is also provided.

### FIELD OF THE INVENTION

The subject matter of the present invention relates generally to carbon dioxide and/or pressure sensing within a tube to verify the placement of the tube in a patient's airway or esophagus.

### BACKGROUND

Physicians and other health care providers frequently use catheters to treat patients. Known catheters include a tube which is inserted into the human body. For instance, some catheters or tubes include endotracheal tubes for delivering mechanical ventilation to a patient's airway. Additionally, certain catheters are inserted through the patient's nose or mouth for treating the digestive or gastrointestinal tract. These catheters, sometimes referred to as enteral catheters, typically include feeding tubes. The feeding tube lies in the stomach or intestines, and a feeding bag delivers liquid nutrient, liquid medicine or a combination of the two to the patient.

When using these known catheters, it is important to place the end of the catheter at the proper location within the human body. However, the esophagus of the digestive tract and the trachea of the respiratory tract are blind to the health care provider during catheter placement. Erroneous placement of the catheter tip may injure or harm the patient. For example, if the health care provider erroneously places an enteral catheter into the patient's trachea, lungs, or other anatomical regions of the respiratory system rather than through the esophagus and to the stomach to reach the desired location in the digestive tract for delivering nutrients or medicine, liquid may be introduced into the lungs with harmful, and even fatal, consequences. In particular, the esophagus of the digestive tract and the trachea of the respiratory system are in close proximity to each other and are blind to the health care provider during catheter placement, which creates a dangerous risk for erroneous catheter placement.

In some cases, health care providers use X-ray machines to gather information about the location of the catheters within the body. There are several of disadvantages with using X-ray machines. For example, these machines are relatively large and heavy, consume a relatively large amount of energy and may expose the patient to a relatively high degree of radiation. Also, these machines are typically not readily accessible for use because, due to their size, they are usually installed in a special X-ray room. This room can be relatively far away from the patient's room. Therefore, health care providers can find it inconvenient to use these machines for their catheter procedures. In addition, using X-ray technology is expensive and is a time-consuming task that can create unnecessary delays in delivering critical nutrients to the patient.

Accordingly, there is a need to overcome each of these disadvantages.

### SUMMARY

Objects and advantages of the invention will be set forth in part in the following description, or may be obvious from the description, or may be learned through practice of the invention.

The present invention is directed to a catheter sensor assembly. The catheter sensor assembly includes a catheter having a proximal end and a distal end and extending in a longitudinal direction, wherein the proximal end and the distal end define a lumen therebetween, and wherein the catheter is configured for placement within a digestive tract or airway of a patient. The catheter sensor assembly also includes an aspiration device and a sensor, wherein the sensor comprises a carbon dioxide sensor, a pressure sensor, or a combination thereof.

In one particular embodiment of the catheter sensor assembly, the sensor can be located at the distal end of the catheter.

In another embodiment, the sensor can be located within the aspiration device.

In an additional embodiment, the sensor can be configured to provide carbon dioxide readings, pressure readings, or a combination thereof measured by the sensor from air in the lumen to a processor in real-time. Moreover, the sensor can be configured for a wired connection or a wireless connection to the processor.

In a further embodiment, the aspiration device can be configured to draw a small volume of air from the lumen of the catheter. Moreover, the aspiration device can be further configured to deliver a positive pressure of air through the lumen of the catheter to the distal end of the catheter. Further, the delivery of positive pressure of air to the distal end of the catheter can be configured to differentiate between placement of the distal end of the catheter in the esophagus and occlusion of the distal end of the catheter when the distal end of the catheter is placed in the airway.

In one more embodiment, the catheter sensor assembly can include a flow rate sensor.

The present invention is further directed to a catheter guidance system comprising: a processor; a power source; a display device, and a catheter sensor assembly. The catheter sensor assembly includes a catheter having a proximal end and a distal end and extending in a longitudinal direction, wherein the proximal end and the distal end define a lumen therebetween; an aspiration device; and a sensor, wherein the sensor comprises a carbon dioxide sensor, a pressure sensor, or a combination thereof. The sensor communicates with the processor via an electrical connection to deliver carbon dioxide readings, pressure readings, or a combination thereof measured by the sensor from air in the lumen to the processor in real-time. The display device is coupled to the processor and displays the carbon dioxide readings, pressure readings, or a combination thereof communicated by the sensor. A carbon dioxide reading profile, a pressure profile, or both a carbon dioxide reading profile and a pressure profile profile after a pre-determined amount of time as shown on the display device indicates placement of the catheter in a digestive tract or an airway of a patient.

In one particular embodiment of the catheter guidance system, the system can include a memory device storing instructions which, when executed by the processor, cause the processor to (i) interpret the carbon dioxide readings, the pressure readings, or a combination thereof communicated by the sensor and (ii) cause the display device to communicate whether the catheter is placed within the digestive tract of the patient or the airway of the patient based on the interpretation of the carbon dioxide readings, the pressure readings, or a combination thereof.

In another embodiment, the sensor can be located within the aspiration device.

In an additional embodiment, the catheter guidance system can further include at least one navigational guide configured to indicate when the distal end of the catheter has passed the epiglottis of the patient when the distal end of the catheter is inserted through the patient's nose or mouth. Moreover, the system can further include a memory device storing instructions which, when executed by the processor, cause the processor to (i) interpret the carbon dioxide readings, the pressure readings, catheter location readings from the at least one navigational guide, or a combination thereof communicated by the sensor and (ii) cause the display device to communicate whether the catheter is placed within the digestive tract of the patient or the airway of the patient based on the interpretation of the carbon dioxide readings, the pressure readings, the catheter location readings, or a combination thereof.

In yet another embodiment, the aspiration device can be configured to draw a small volume of air from the lumen of the catheter to deliver a positive pressure of air through the lumen of the catheter to the distal end of the catheter.

The present invention is further directed to a method for determining if a catheter is placed within a digestive tract or an airway of a body of a patient. The method include a step of inserting a distal end of a tubing assembly into an orifice of the body. The catheter sensor assembly includes: the catheter, wherein the catheter has a proximal end and a distal end and extends in a longitudinal direction, wherein the proximal end and the distal end define a lumen therebetween; an aspiration device; and a sensor, wherein the sensor comprises a carbon dioxide sensor, a pressure sensor, a flow sensor, or a combination thereof. The method further includes a step of activating the sensor, wherein the sensor measures carbon dioxide, pressure, or a combination thereof from air in the lumen and communicates with the processor via the wired connection or the wireless connection to deliver carbon dioxide readings, pressure readings, or a combination thereof to the processor in real-time, wherein a display device is coupled to the processor and displays the carbon dioxide readings, pressure readings, or a combination thereof communicated by the sensor. The method further includes steps of advancing the distal end of the catheter inside the body in a direction away from the orifice while the sensor is activated; and observing the carbon dioxide readings, pressure readings, flow readings, or a combination thereof on the display device, wherein a carbon dioxide reading profile, a pressure reading profile, a flow reading profile, or a combination of a carbon dioxide reading profile, a pressure reading profile and/or a flow reading profile after a pre-determined amount of time indicates placement of the catheter in a digestive tract or an airway of a patient.

In one particular embodiment of the method, a memory device stores instructions which, when executed by the processor, cause the processor to (i) interpret the carbon dioxide readings, the pressure readings, or a combination thereof communicated by the sensor and (ii) cause the display device to communicate whether or not the catheter is placed within the digestive tract of the patient based on the interpretation of the carbon dioxide readings, the pressure readings, the flow readings, or a combination thereof.

In one embodiment, the orifice can be a nose or a mouth.

In another embodiment, the sensor can be located within the aspiration device.

In a further embodiment, suction from the aspiration device can direct air sampled from a distal end of the catheter to the sensor. Moreover, the aspiration device can deliver at least one puff of positive air pressure to the distal end of the catheter then resumes suction of air from the distal end of the catheter to determine if the distal end of the catheter is located within the esophagus or if the distal end of the catheter is located within the airway and occluded.

In an additional embodiment, the method can include a step of delivering a positive pressure of air from the aspiration device through the distal end of the catheter while inserting the distal end of the catheter inside the body in a direction away from the orifice until the distal end of the catheter reaches a predetermined anatomical reference point. Moreover, steps (b) and (c) can be performed after the distal end of the catheter reaches the predetermined anatomical reference point.

In yet another embodiment, the method can include a step of: providing at least one navigational guide, wherein information from the at least one navigational guide is configured to indicate placement of the catheter in a digestive tract or an airway of a patient. Moreover, a memory device stores instructions which, when executed by the processor, cause the processor to (i) interpret the carbon dioxide readings, the pressure readings, the information from the at least one navigational guide, or a combination thereof and (ii) cause the display device to communicate whether or not the catheter is placed within the digestive tract of the patient based on the interpretation of the carbon dioxide readings, the pressure readings, the flow readings, the information from the navigational guide or a combination thereof.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a perspective view of the catheter guidance system illustrating the display device, catheter unit and the sensor that is at least temporarily in communication with the catheter unit as it is being used to position a catheter within a patient in one embodiment of the present invention.

FIG. 2 is schematic block diagram of the electronic configuration of the catheter position guidance system illustrating the processor, memory device, sensor, input devices, and output devices in one embodiment of the present invention.

FIG. 3 is a perspective view of the catheter unit illustrating the catheter sensor assembly having a tubing assembly, aspiration line and aspiration device according to various embodiments of the present invention.

FIG. 4A is a perspective view of a sensor assembly portion of an electronic catheter unit according to one embodiment of the present invention.

FIG. 4B is a perspective view of the sensor assembly portion of the electronic catheter unit within the airway sampling chamber according to one embodiment of the present invention.

FIG. 5A is a perspective view of the aspiration device according to one embodiment of the present invention.

FIG. 5B is a schematic block diagram of the electronic configuration of one embodiment of the aspiration device of the present invention.

FIG. 5C is a schematic block diagram of the electronic configuration of an additional embodiment of the aspiration device of the present invention.

FIG. 6A is a top or plan view of a portion of the electronic catheter unit illustrating an enteral application involving insertion of a catheter into the esophagus of a patient, where the anatomical location of the catheter within the body can be monitored or traced via the sensor assembly of the present invention.

FIG. 6B is a schematic view of the catheter guidance system of the present invention as the system measures the carbon dioxide level of air sampled from the catheter of FIG. 6A in real-time via the sensor assembly.

FIG. 6C is a schematic view of the catheter guidance system of the present invention as the system measures the pressure of air sampled from the catheter of FIG. 6A in real-time via the sensor assembly.

FIG. 7A is a top or plan view of a portion of the electronic catheter unit illustrating an enteral application involving insertion of a catheter erroneously into the lung of a patient, where the anatomical location of the catheter within the body can be monitored or traced via the sensor assembly of the present invention.

FIG. 7B is a schematic view of the catheter guidance system of the present invention as the system measures the carbon dioxide level of air sampled from the catheter of FIG. 7A in real-time via the sensor assembly.

FIG. 7C is a schematic view of the catheter guidance system of the present invention as the system measures the pressure of air sampled from the catheter of FIG. 7A in real-time via the sensor assembly.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present invention. For example, if ranges of “from about 20% to about 80%” and “from about 30% to about 70%” are described, a range of “from about 20% to about 70%” or a range of “from about 30% to about 80%” are also contemplated by the present invention.

Generally speaking, the present invention is directed to a tubing assembly that includes a catheter having a proximal end and a distal end and extending in a longitudinal direction, where the proximal end and the distal end define a lumen therebetween. Further, the catheter is configured for placement within a digestive tract or an airway of a patient. The tubing assembly also includes a sensor, where the sensor includes a carbon dioxide sensor, a pressure sensor, a flow sensor, or a combination thereof. The sensor can be located within the lumen of the catheter or in an air sampling chamber connected to the catheter. The sensor can communicate with a processor to deliver carbon dioxide and/or pressure readings to a display device. A catheter guidance system and a method for accurately placing a catheter in the digestive tract are also contemplated by the present invention.

The present inventors have found that the tubing assembly, catheter guidance system, and method described in more detail herein allow for the continuous sampling of air during an intubation procedure of a patient, independently of inspiration or expiration of the patient, where the real-time carbon dioxide and/or pressure readings measured by the sensor can be used to determine if the distal end of the catheter is placed within the digestive tract (e.g., esophagus, stomach, intestines, etc.) or within the respiratory system (e.g., trachea, bronchi, lungs, etc.), in order to prevent improper placement that could be harmful and even fatal to a patient. Further, the present inventors have found that because the sensor can obtain measurements and communicate those measurements to processor and ultimately a display device or other communication device (e.g., a phone, pager, etc.) in real time, the correct placement of the catheter can be confirmed within seconds of a catheter placement procedure, which can save valuable time, resources, and cost while at the same time limit patient risk in the event of the erroneous placement of the catheter.

Specifically, the present inventors have found that the real-time monitoring of the carbon dioxide and the pressure and/or flow of the air inside or within a catheter to be placed in a predetermined location along the digestive tract (e.g., esophagus, stomach, intestines, etc.) or respiratory tract (e.g., trachea), which is facilitated by the sensor assembly of the catheter guidance system of the present invention, allows for the efficient and accurate placement of the catheter within the intended portion of the patient's anatomy at a low cost. For instance, the sensor in communication with the tubing assembly can monitor the carbon dioxide level and/or pressure and/or flow of air within the catheter as it is being directed by a health care provider in to the body of a patient, where the carbon dioxide, pressure and/or flow data can be transmitted to a display device via a processor. The health care provider can then view the carbon dioxide, pressure and/or flow data to determine if the catheter has been accurately placed, e.g., in the digestive tract, or erroneously placed, e.g., in an anatomical region of the respiratory system (e.g., the trachea, bronchi, lungs, etc.). Alternatively or additionally, a memory device that can include machine readable instructions and one or more computer programs (which, for example, may include a plurality of algorithms) can be used by the processor to process the data from the sensor, where the display device can then indicate the catheter information to the health care provider in the form of a signal as to whether the catheter is accurately placed, e.g., in the digestive tract, or erroneously placed, e.g., within a portion of the respiratory system. For example, a green check mark or the word “Yes” can be displayed on the screen to indicate accurate placement of the catheter within the digestive or gastrointestinal tract, while a red circle with a diagonal line through it, an “X”, or the word “No” can be displayed on the screen for erroneous placement, such as placement within the respiratory system.

The various features of the catheter guidance system are discussed in detail below.

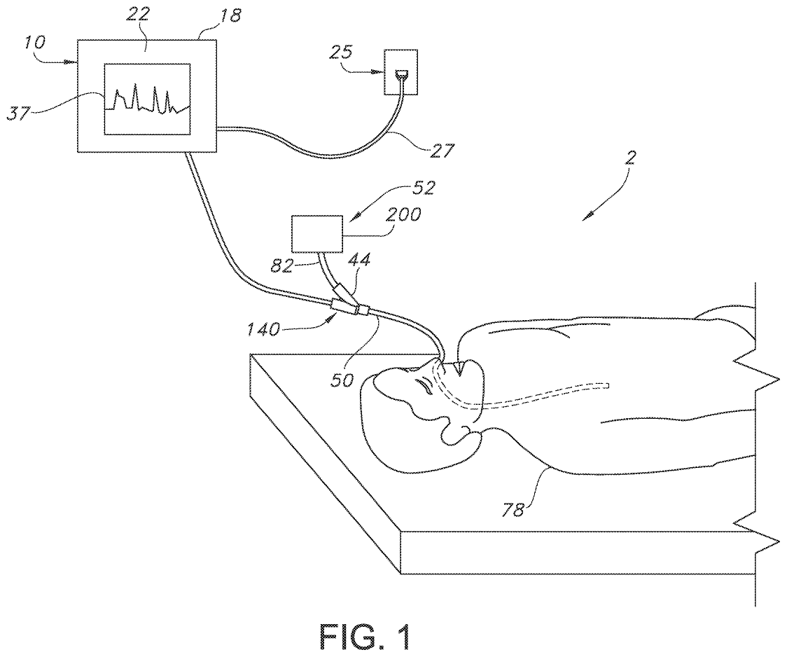

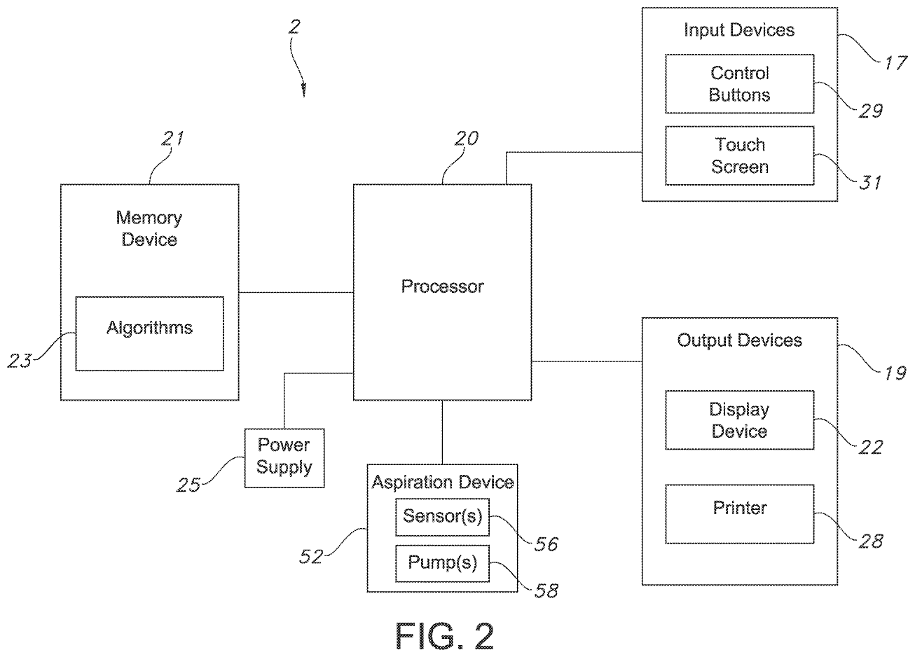

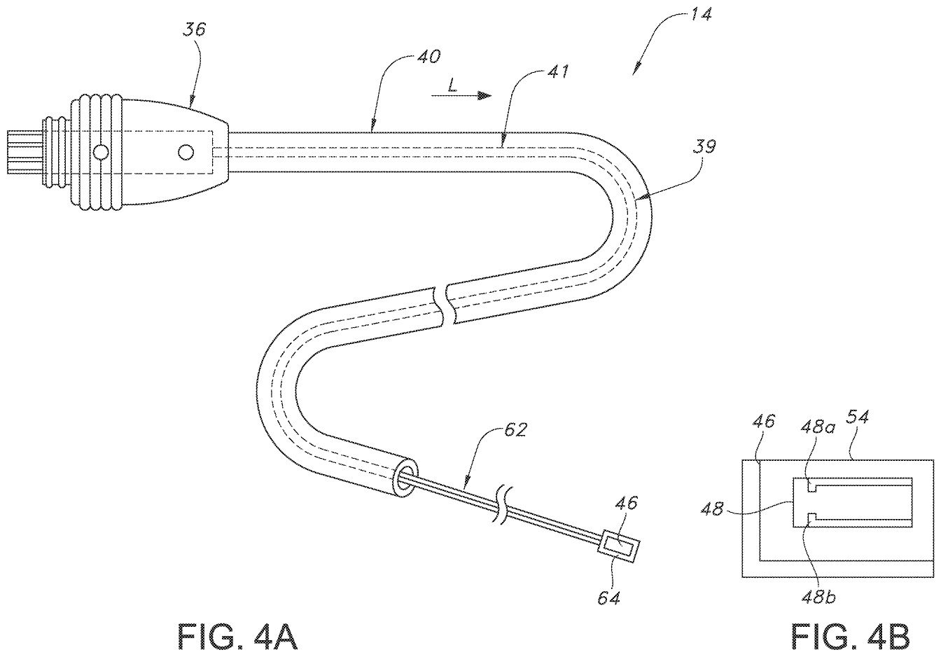

Referring now to the drawings, in an embodiment illustrated in FIGS. 1-4B, the catheter guidance system 2 contemplated by the present invention includes: (a) an apparatus 10 having a housing 18 which supports a controller or processor 20 (see FIG. 2) and a display device 22; (b) a power cord 27 that couples the apparatus 10 to a power source 25; (c) optionally, a printer 28 (see FIG. 2) coupled to the apparatus 10 for printing out paper having graphics which indicate catheter location information; and (d) a catheter unit 12 in communication with and operatively coupled to the apparatus 10, where the catheter unit 12 includes a tubing assembly 14 that includes a catheter 50 and optionally a sensor 46. As shown in FIG. 4A, in an embodiment in which the catheter unit includes the sensor 46, the catheter unit 12 may be operatively coupled to the apparatus 10 by a wire, cable, cord or electrical extension 34, which, in turn, is operatively coupled to the processor 20.

As best illustrated in FIG. 2, the system 2, in one embodiment, includes: (a) a plurality of input devices 17 for providing input signals to the system 2 such as one or more control buttons 29, a touch screen 31, etc.; (b) an aspiration device 52 having one or more sensor(s) 56 that can continuously measure the carbon dioxide level and/or pressure and/or flow of air inside or within a catheter 50 of the tubing assembly 14 in real-time; (c) a memory device 21 including machine readable instructions and one or more computer programs (which, for example, may include a plurality of algorithms 23) which are used by the processor 20 to process the signal data produced by the sensor(s) 56; and (d) a plurality of output devices 19 such as the display device 22 and the printer 28 which indicate the catheter information to the health care provider, such as in the form of a graph 37 (see FIGS. 1, 6B, and 7B). The display device 22 may be any suitable display mechanism including, but not limited to, a liquid crystal display (LCD), light-emitting diode (LED) display, cathode-ray tube display (CRT) or plasma screen.

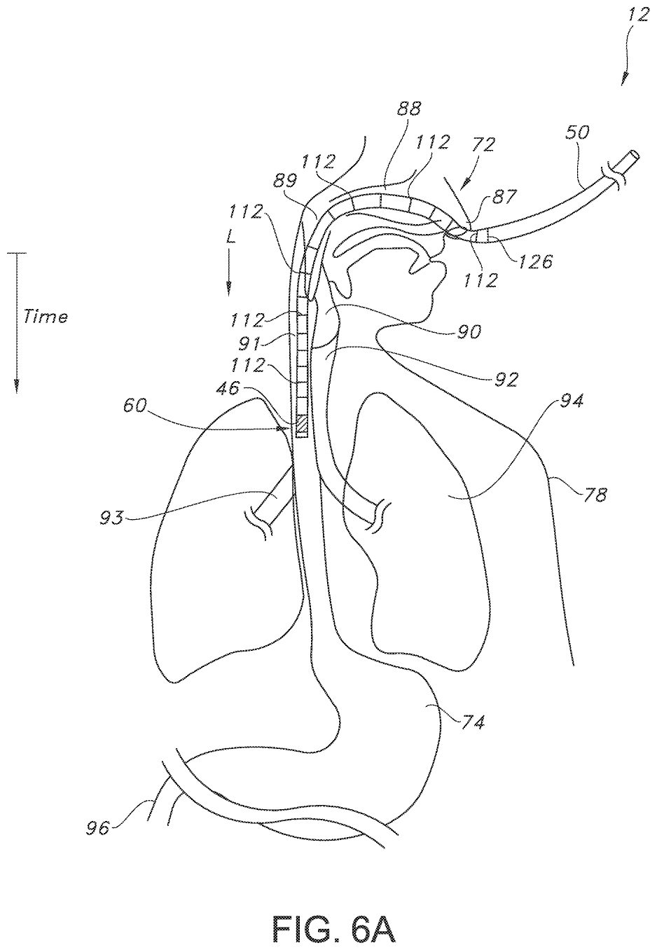

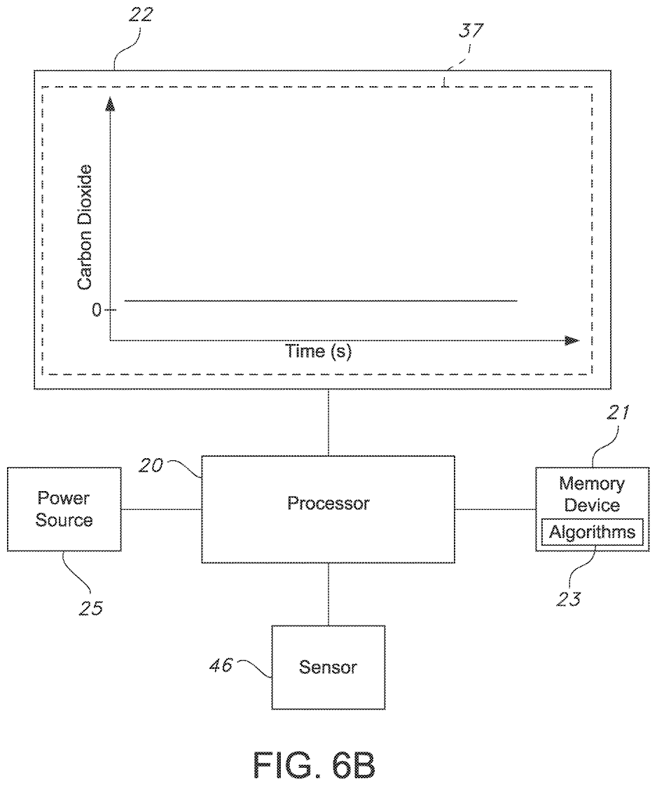

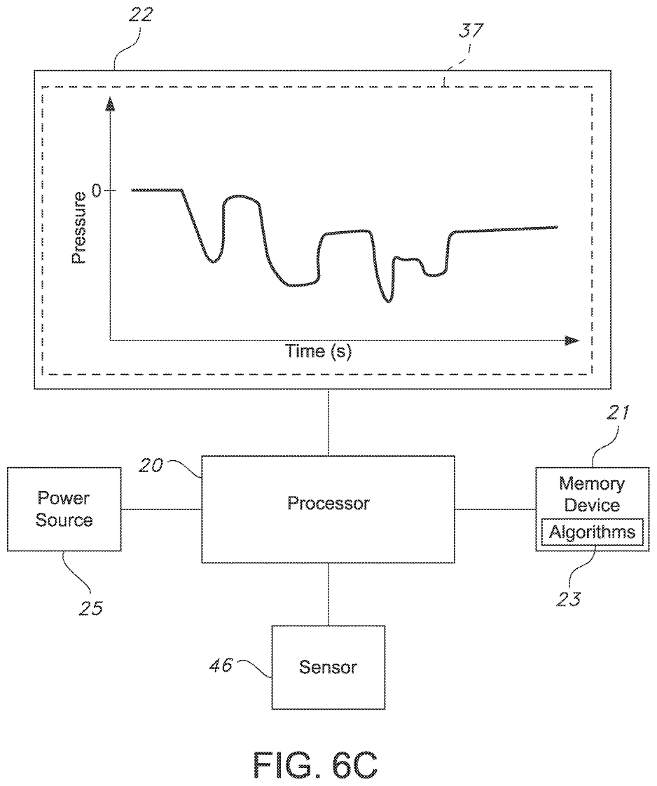

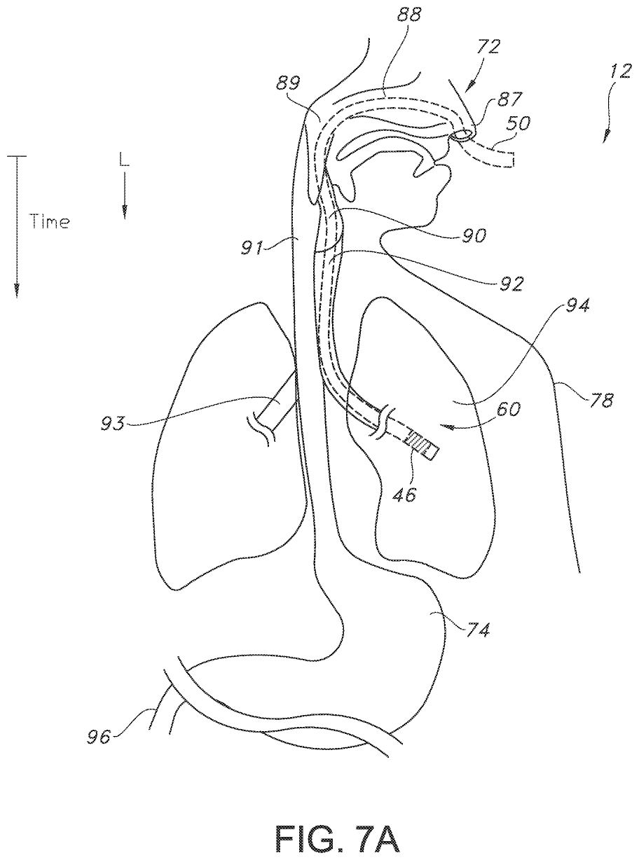

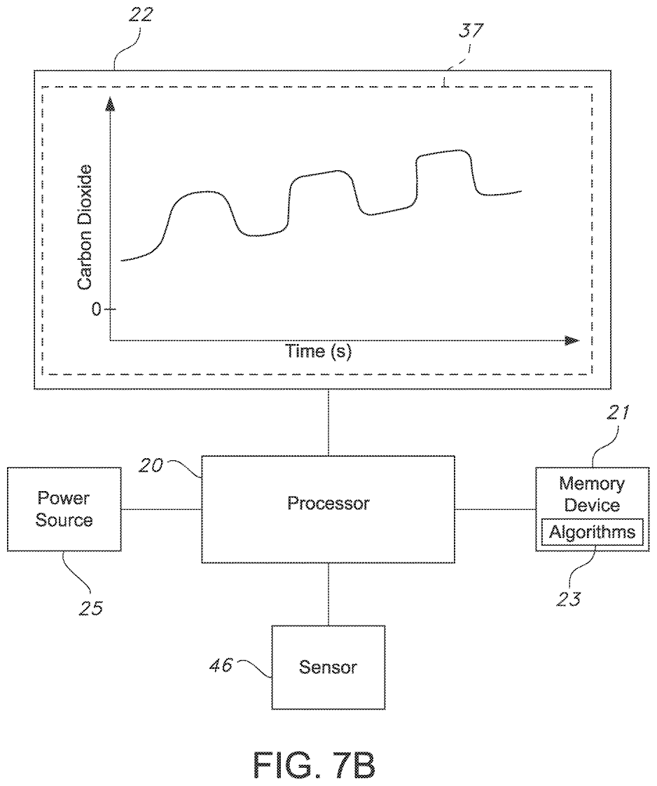

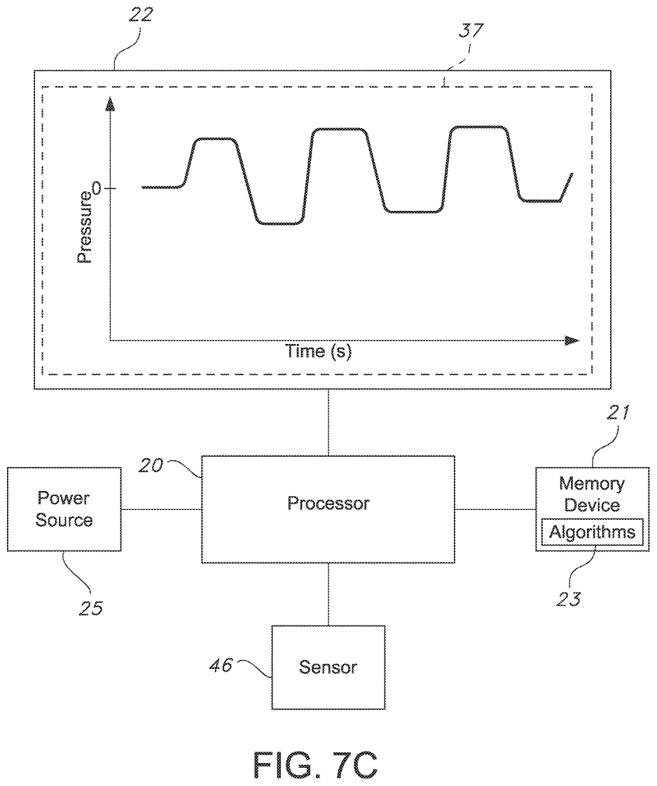

Health care providers can use the system 2 in a variety of catheter applications. In one example illustrated in FIGS. 6A and 7A, the system 2 is used in an enteral application. Here, a portion 70 of the electronic catheter unit 12 is placed through an orifice 72 of the patient, such as the patient's nose or mouth. The distal end or tip 60 of the electronic catheter unit 12 can ultimately by positioned in the stomach 74. As the health care provider advances the catheter 50 of the electronic catheter unit 12 towards the patient's stomach, a sensor 46 within the catheter 50 and/or the sensor(s) 56 within the aspiration device 52 in communication with the catheter 50 can continuously monitor the carbon dioxide level and/or pressure and/or flow of the air sampled within the catheter 50 and drawn into the aspiration device 52 as shown in FIGS. 1 and 4. The display device 22 and the printer 28 can indicate information related to the location of the portion 70 of the electronic catheter unit 12 within the body 78, as well as information related to the shape of the pathway taken by the catheter unit 12. It should be appreciated that the system 2 need not indicate the exact location or path of the catheter unit 12 to provide assistance to the health care provider.

Referring to FIG. 4A, in one embodiment, the catheter unit 12 includes a tubing assembly 14, which includes the catheter 50 and a sensor 46 disposed within the catheter 50, where the catheter 50 can generally extend in the longitudinal direction L. In one embodiment, the catheter unit 12 can include an aspiration device 52, shown in FIGS. 1, 3 and 5A, that can house one or more sensor(s) 56. However, it is also to be understood that the sensor 46 can be located anywhere along the length of the catheter 50. In another embodiment, the sensor 46 can be disposed within the lumen 70 of the catheter 50 at a distal end or tip 60 of the catheter 50. Together, the tubing assembly 14, the aspiration device 52 and the sensor(s) 46, 56 can form a catheter sensor assembly.

As best illustrated in FIGS. 1 and 4A, in one embodiment, such as when a wired connection (as opposed to a wireless connection, which is also contemplated by the present invention, where the sensor 46 includes a battery or other source of power) electrically connects the sensor 46 to the processor 20, the tubing assembly 14 can include (a) a tube or an electrical tubular insulator 40; (b) a mid-connector or union device (not shown) which receives the tubular insulator 40; (c) a multi-port connector or y-port connector 44 attachable to the union device; (d) a catheter 50, such as a feeding tube, connected to the y-port connector 44; and (e) the distal end or tip 60 of the catheter 50, where the sensor 46 can be located within the lumen 70 of the catheter 50 at the distal end or tip 60 or anywhere upstream along the length of the catheter 50.

In one embodiment, the tubular insulator 40 includes a tube having a proximal end attachable to an attachment member or neck of a controller coupler or electrical connector 36 and a distal end receivable by the union device; and an internal diameter which is substantially equal to or greater than an external diameter of a wire assembly 62 described below, which can serve as the hard wired electrical connection between the sensor 46 and the processor 20, so as to slide over the wire assembly 62. In another embodiment, the tubular insulator 40 may fit relatively tightly over the wire assembly 62 so as to be secured to the wire assembly 62.

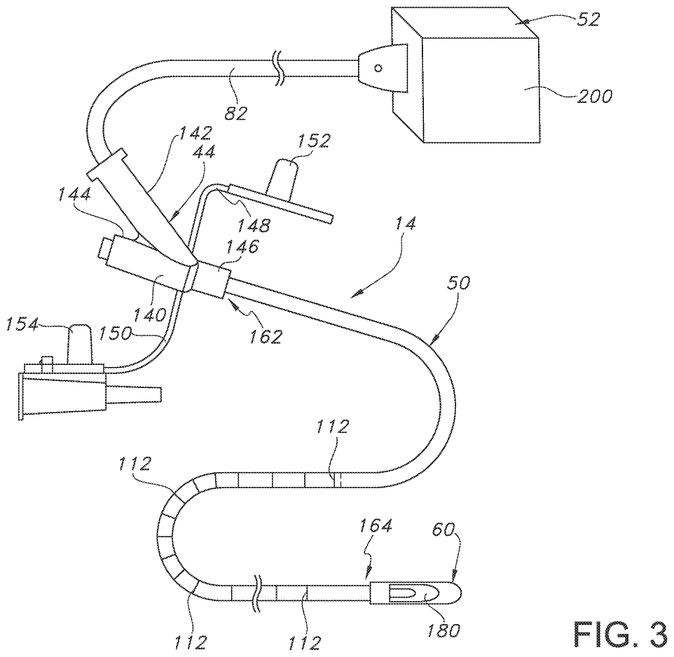

In one embodiment best shown in FIG. 3, the multi-port or y-port connector 44 includes: (a) a body 140; (b) a liquid delivery branch, medicine delivery branch or medicine branch 142 attached to the body 140 for distributing drugs, medicine or other medicinal liquids to the patient; (c) a nutrient delivery branch or feeding branch 144 attached to the body 140 and sized to receive the insert 124 of the union device 42; (d) a catheter or feeding tube connection branch 146 attached to the catheter 50; (e) a flexible or movable arm 148 attached to the body 140; and (f) a flexible or moveable arm 150 attached to the body 140. In an alternative embodiment, y-port connector 44 includes additional branches for administering various nutrients or medicines to the body 78. In another alternative embodiment, the y-port connector 44 includes only a feeding branch 144 and a connection branch 146. The arm 148 has a stopper 152, and the arm 150 has a stopper 154. The stoppers 152 and 154 are sized to prevent fluid from passing through the branches 142 and 144 after such branches 142 and 144 are plugged with stoppers 152 and 154, respectively. In addition, the arm 150 includes a fastener 155 which secures a tube-size adapter 156 to the arm 150. The tube-size adapter 156 enables fluid delivery tubes (not shown) having various diameters to connect to the feeding branch 144 of the y-port connector 44.

As illustrated in FIG. 3, in one embodiment, the catheter 50 includes a feeding tube or catheter 50 with a body 160 having a proximal end 162 attached to the catheter connection branch 146 of the y-port connector 44 and a distal end 164. The proximal end 162 is insertable into the catheter connection branch 146 of the y-port connector 44 so as to bring the catheter 50 into fluid communication with the y-port connector 44.

As also shown in FIG. 3, in one embodiment, the end member, bolus or tip 60 is attached to the distal end 164 of the catheter 50. The tip 60 includes an opening 180. The shape of the opening 180 of the tip 60 is configured to facilitate the flow of fluid from the catheter 50 into the patient's body while decreasing the likelihood that the opening 180 will become clogged.

The tubular connector 40, y-port connector 44, catheter 50, and tip 60 can be made from any suitable polymer or plastic material including, but not limited to, polyamide, polyethylene, polypropylene, polyurethane, silicone and polyacrylonitrile.

Referring still to FIGS. 1-3 and 5A, in some embodiments, the tubing assembly 14 can be connected to an aspiration device 52 that can help in drawing air through the catheter 50 and/or provide a housing for one or more sensor(s) 56 that can be exposed to a continuous flow of air for measuring the carbon dioxide and/or pressure of the sample of air in real-time. For instance, the aspiration device 52 can be connected to the catheter 50 by one or more aspiration lines 82, or alternative the aspiration device 52 can be connected directly to the catheter 50 at the distal end (not shown). Another possible location for the aspiration line 82 can be attached to the delivery branch or medicine branch 142 of the multi-port connector or y-port connector 44, such as when the sensor(s) 56 are located in the aspiration device 82 rather than having a sensor 46 in the lumen 70 of the catheter. In such an arrangement, the aspiration device 82 can be connected to the aspiration line 82, where the sensor is then electrically connected to the processor 20

Turning now to the specifics of the sensor 46 and referring to FIGS. 1 and 4A-B, a controller coupler or an electrical connector 36 can be operatively connected to the electrical extension 34 and an elongated wire assembly 62 can be operatively coupled to the connector 36 to form a wired connection between the sensor 46 and the processor 20, although it is to be understood that the electrical connection between the processor 20 and the sensor 46 can also be wireless provided that the sensor 46 has its own power source, such as a battery. Further, a wire or elongated stiffener 39 can be attached to the connector 36 and can serve as a support for the wire assembly 62 when it is inserted into the body 160 of the catheter or the tubing 66. Further, the tubular insulator 40 described above can cover a portion 41 of the wire assembly 62 positioned adjacent to the connector 36 in the embodiment where the sensor 46 is positioned within the lumen 70 of the catheter 50. In any event, the electrical connector or controller coupler 36 can provide the electrical connection between the apparatus 10 and the sensor 46 when the sensor 46 is hard wired to the catheter guidance system 2 via the wire assembly 62, regardless of whether the sensor 46 is positioned within the lumen 70 of the catheter or within the air sampling chamber 54.

When the sensor 46 is disposed within the lumen 70 of the catheter 50, the sensor 46 can be surrounded by a filter formed from a porous filter material or porous filter media in order to prevent moisture from the opening 180 in the tip 60 of the catheter 50 from contacting the sensor 46 and affecting its carbon dioxide and/or pressure or flow readings. For instance, the filter can prevent water or other fluid ingress that may enter through the opening 180 from contacting the sensor 46, while still allowing air to penetrate into the lumen 70. In any event, the filter 64 is positioned within the tubing assembly 14 to protect the sensor 46 from water or other fluid ingress that may damage the sensor 46 of affect the accuracy of its carbon dioxide, pressure and/or flow readings.

Turning now to the makeup of the filter, the filter contemplated by the present invention can allow gases but not liquids to pass therethrough. Stated alternately, the filter of the present invention can be vapor permeable and liquid impermeable. The filter may comprise any suitable material or combination thereof. Exemplary suitable materials for the filter include but are not limited to reticulated polymer foams, expanded polymers (such as Porex® expanded polymers available from Porex Corporation, having offices in Fairburn, Ga.), expanded PTFE (such as Gore-Tex® expanded PTFE available from W. L. Gore & Associates, Inc., having offices in Newark, Del.), and porous metals (or powdered metals). As will be appreciated, the rate at which the gases are allowed to pass through the filter is not critical so long as it is sufficient to allow for a sufficient volume of air to come into contact with the sensor 46 to obtain accurate carbon dioxide, pressure and/or flow readings. It will also be appreciated that air flow rate may be affected or controlled in part by the composition of the filter. Nevertheless, in most embodiments, it is generally desirable for the insert to be able to allow at least 3 liters to 5 liters of gas to pass therethrough per hour. For use with a pediatric catheter, it may be desirable for the filter in an appropriately sized adapter to be able to allow at least 1 liter to 2 liters of gas to pass therethrough per hour. Further, it will be appreciated that the filter 64 may be hydrophobic or hydrophilic, although it is desired that the insert or insert media be generally hydrophobic. Where the filter is or contains a hydrophobic filter media or where the filter media is at least in part hydrophobically treated, the filter media may have larger pore sizes and therefore a higher flow rate therethrough (as compared to a hydrophilic or hydrophilically treated media) as the filter will be less likely to absorb liquids, become saturated and allow liquid to pass therethrough.

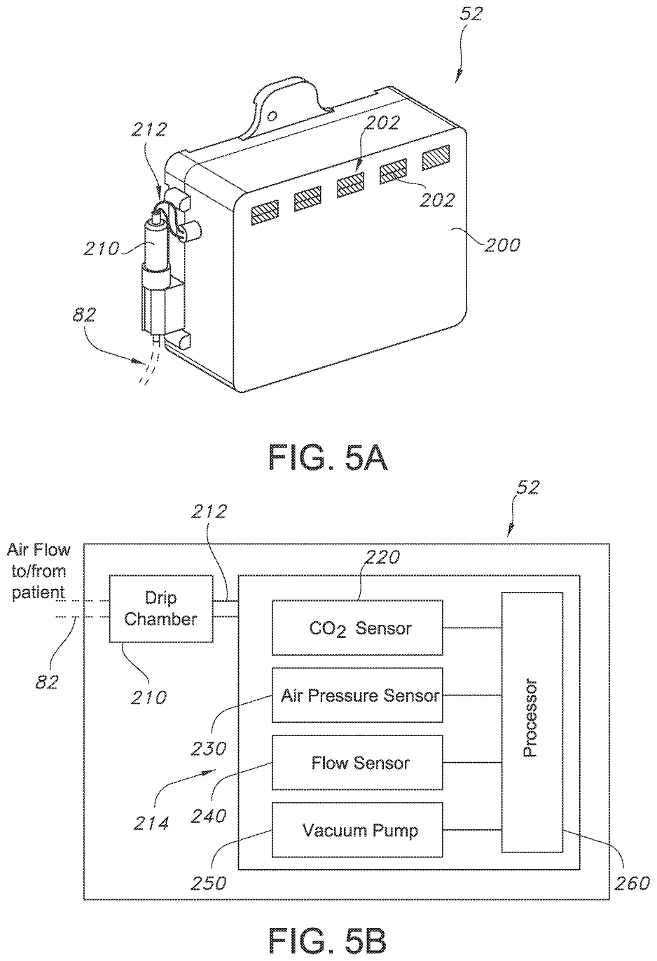

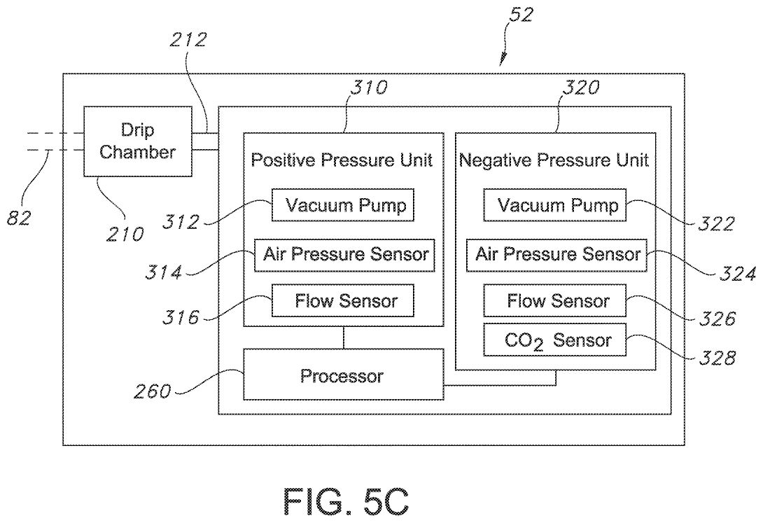

As shown in FIGS. 1, 3 and 5A, the aspiration device 52 may be connected to the catheter 50 via an aspiration line 82. The aspiration device 52 may include a housing 200. In some embodiments, the aspiration device 52 includes a drip chamber 210 disposed between the aspiration line 82 and an inlet/outlet tube 212 of the housing 200. The drip chamber 210 is configured to collect fluid, mucus, or any other liquid or solid matter that is pulled into the aspiration line 82 and prevent any liquid or solid matter from clogging the inlet/outlet tube 212. Although the drip chamber 210 is shown mounted to the exterior of the housing 200 in FIG. 5A, it is contemplated that the drip chamber 210 could be disposed anywhere between the catheter 50 and the housing 200 or within the housing 200 itself. The housing 200 of the aspiration device 52 may further include one or more apertures 202 for enabling air flow in and out of the housing 200.

FIG. 5B illustrates a schematic block diagram of one embodiment of aspiration device 52. The aspiration device 52 includes an optional drip chamber 210, as described above, and an inlet/outlet tube 212 connected to an aspiration and sensing unit 214. The aspiration and sensing unit 214 includes at least one pump 58, such as the vacuum pump 250 shown in FIG. 5B. The vacuum pump 250 can be used to generate a negative pressure or vacuum through the catheter 50 in order to draw air from the distal tip 60 of the catheter 50 into the aspiration device 52 in order to be able to sense the carbon dioxide, air pressure and/or flow of the air at the tip 60 of the catheter 50. The vacuum pump 250 can additionally be used to generate positive pressure delivered to the catheter 50 through the inlet/outlet tube 212, e.g., to clear the inlet/outlet tube 212 and the catheter 210 of liquid or solid secretions during insertion of the catheter 50 into the patient's body 78. For instance, positive pressure can be generated by the vacuum pump 250 and delivered to the catheter 50 to assist with the insertion and placement of the catheter 50 in the body, e.g., insertion into the small intestine. The aspiration and sensing unit 214 additionally includes at least one sensor 56, such as a carbon dioxide (CO2) sensor 220, an air pressure sensor 230, and/or a flow sensor 240. In a preferred embodiment, the aspiration and sensing unit 214 includes each of the carbon dioxide (CO2) sensor 220, the air pressure sensor 230, and the flow sensor 240. Additionally, the aspiration device 52 can include a processor 260 configured to control the pump 250 and the sensors 220, 230, 240 and/or communicate with the processor 20 of the device 2.

FIG. 5C illustrates a schematic block diagram of another embodiment of the aspiration device 52 in which the positive pressure and negative pressure functions are separated into a positive pressure unit 310 and a negative pressure unit 320. The negative pressure unit 320 can include its own vacuum pump 322, an air pressure sensor 324, a flow sensor 326, and a carbon dioxide (CO2) sensor 328 in order to be able to sense the carbon dioxide, air pressure and/or flow of the air drawn in from the distal tip 60 of the catheter 50. The positive pressure unit 310 can include a vacuum pump 312 for generating a positive air pressure to be delivered into the catheter 50, an air pressure sensor 314 and a flow sensor 316. Both the positive pressure unit 310 and the negative pressure unit 320 can be operatively connected to the processor 260 of the aspiration device 52.

Additionally, although any suitable sensor(s) 46, 56 for measuring carbon dioxide and pressure and/or air flow that can withstand the environmental conditions of the body can be used in the catheter guidance system 2, the sensor(s) 46, 56 can be in the form of a flip chip package having a small footprint such that it can be placed within the housing 200 of the aspiration device 52, lumen of the catheter 50, or any other suitable location within the tubing assembly 14. For instance, the sensor(s) 46, 56 can include a digital carbon dioxide sensor and a digital pressure and/or volumetric air flow sensor that includes analog and digital signal processing, an A/D converter, calibration data memory, and a digital communication interface for communication with the processor 20, all of which combine to allow for real-time, continuous, and highly accurate carbon dioxide and pressure and/or air flow sensing.

For instance, the carbon dioxide sensor can be an infrared carbon dioxide sensor or any other suitable type of capnograph or carbon dioxide sensor. The sensor(s) 46, 56 can include an MEMS component 48 having one or more MEMS active and passive components that form a non-dispersive infrared (IR) sensor. Carbon dioxide (CO2) strongly absorbs infrared radiation at a wavelength of 4.3 μm. Further, the carbon dioxide concentration at the end of a person's exhaled breath is approximately 5% to 6% of the exhaled air, which corresponds to about 35 mmHg to about 45 mmHg. Therefore, the MEMS infrared sensor is configured to detect carbon dioxide to determine whether the catheter 50 is being placed in the patient's airway and may be referred to as a MEMS infrared carbon dioxide sensor 48. More particularly, the MEMS component 48 includes an IR emitter 48a and an IR receiver 48b, which form the MEMS infrared carbon dioxide sensor 48. The IR emitter 48a emits infrared radiation, and the IR receiver 48b receives any reflected radiation. An IR path length between the IR emitter 48a and the IR receiver 48b dictates the carbon dioxide concentration the IR carbon dioxide sensor 48 can detect. Thus, the MEMS component 48, particularly the IR emitter 48a and IR receiver 48b, should be constructed such that the sensor can detect a carbon dioxide concentration of at least 30 mmHg to 50 mmHg and, in particular embodiments, of at least 35 mmHg to 45 mmHg. The IR carbon dioxide sensor can be disposed within the catheter 50 or the aspiration device 52.

The carbon dioxide sensor may generate an electrical signal corresponding to the level of carbon dioxide sensed by the sensor(s) 46, 56, and the voltage level of the signal varies based upon the level of carbon dioxide sensed by the sensor 46. In addition, the sensor(s) 46, 56 can also have a low operation voltage of less than 2.5 volts, such as from about 0.5 volts to about 2 volts, such as from about 1 volt to about 1.9 volts, such as about 1.8 volts, which allows for low power consumption, which can allow for the sensor(s) 46, 56 to be suitable for applications where the electrical connection between the sensor(s) 46, 56 and the processor 20 is wireless as opposed to a wired connection via the wire assembly 62, although a wired connection between the sensor 46 and the process 20 via the electrical connector or controller coupler 36 is still possible.

The carbon dioxide (CO2) concentration at the end of a person's exhaled breath is approximately 5% to 6% of the exhaled air, which corresponds to about 35 mmHg to about 45 mmHg. The sensor(s) 46, 56 determines the carbon dioxide concentration of the air within the air from the lumen 70, such as the air drawn into the catheter 50. In some embodiments, if the carbon dioxide concentration is at least 30 mmHg, the system 2 may determine that the tip 60 of the catheter 50 is placed in the patient's airway. In other embodiments, the system 2 may determine that the tip 60 of the catheter 50 is being placed in the patient's airway if the carbon dioxide concentration is at least 35 mmHg. That is, sensor(s) 46, 56 can be configured to sense a carbon dioxide concentration of at least 30 mmHg, or in other embodiments, of at least 35 mmHg, which corresponds to the low end of the typical range of carbon dioxide concentration in a person's exhaled breath. When the sensor(s) 46, 56 senses such a carbon dioxide concentration, the sensor(s) 46, 56 may provide feedback of the carbon dioxide concentration to the user via the display 22. In some embodiments, the feedback from the sensor(s) 46, 56 indicates the tip 60 is entering the airway when the carbon dioxide concentration sensed by the sensor 46 is 30 mmHg or 35 mmHg. In other embodiments, if the carbon dioxide concentration continues to rise past 30-35 mmHg as the catheter 50 is advanced into the patient, as shown on the display 22, the user may determine that the catheter 50 is being incorrectly placed in the patient's airway because the rising carbon dioxide concentration likely corresponds to the patient's respirations conveyed through the patient's airway. Stated differently, using the carbon dioxide level or concentration that is detected by the sensor(s) 46, 56, the user can determine whether the distal tip 60 of the catheter 50 resides in the patient's airway.

The pressure readings of the sensor(s) 46, 56 as the catheter 50 is inserted into either the digestive tract, e.g., esophagus, or the respiratory tract, e.g., trachea, may be used to determine placement of the catheter 50 based on anatomical differences between the esophagus and the trachea. For example, the esophagus contains no significant structure support and readily collapses when negative pressure is applied. Conversely, the trachea is lined with semi-rigid cartilage that maintains patency in the airway, even under moderate negative pressure. Thus, applying a negative pressure through a tube or catheter, including through the inner lumen of the nasogastric/nasojejunal tube, during placement can differentiate the location of the catheter or tube's tip based on this anatomical difference.

The aspiration device 52 can additionally be used to deliver a positive pressure of air through tip 60 of the catheter 50. For instance, during insertion of the catheter 50 into the patient's body 78, the aspiration device 52 can deliver a positive pressure of air through the tip 60 of the catheter in order to prevent any liquid, mucus, food particles, or other secretions from entering and/or clogging the tip 60 of the catheter 50. Additionally or alternatively, as will be described in more detail below, the aspiration device 52 can deliver one or more puffs of positive air pressure to assist with differentiating between placement of the catheter 50 in the digestive tract as compared to the respiratory tract, and to assist with determining whether the tip 60 of the catheter 50 is occluded. Moreover, the aspiration device 52 can be used to deliver positive pressure of air through the tip 60 of the catheter 50 to assist with insertion of the catheter 50 into the patient's body 78, such as insertion into the small intestine.

In some aspects of the invention, the catheter guidance system 2 can include a navigational guide for determining the depth of placement of the tip 60 of the catheter 50 within the patient's body. For instance, in one embodiment and referring to FIGS. 3 and 6A, the catheter body 160 can have a plurality of markings 112 uniformly spaced along its external surface that can be used in conjunction with the sensor(s) 56 of the aspiration device 52 to determine accurate placement of the catheter 50. These markings 112 can function as placement markers which assist the user in assessing the depth that the catheter 50 is placed within the body 78 in order to identify when the catheter has likely reached a desired anatomical reference point. For instance, the markings 112 can be present from the distal end 60 of the catheter 50 to a point 126 on the catheter 50 that spans a distance that can correspond with the average distance between the epiglottis 90 and nostril 87 in a typical patient. As the catheter 50 is being inserted into the body 78 via the nostril 87, once the markings 112 are no longer visible outside the body 78, the user can be alerted to start looking carbon dioxide reading profile and/or pressure and/or flow reading profile as measured by the sensor(s) 46, 56. If the carbon dioxide readings are still oscillating to the analog of breathing once the markings 112 are no longer visible outside the body 78, then the user will be able to determine that the catheter 50 has been inserted into the trachea 92 instead of the esophagus 91. Similarly, if the pressure readings are a generally constant or decreasing negative pressure, then the user will be able to determine that the catheter 50 has been inserted into the esophagus 91 rather than the trachea 92. In an alternative embodiment, these markings 112 can assist the user in measuring the flow or distribution of liquid to or from the patient.

Additionally or alternatively, the catheter guidance system 2 can be used with an electromagnetic catheter position guidance system (not shown) that can function as a navigational guide. The electromagnetic catheter position guidance system may include one or more electromagnetic transmitter(s) and/or receiver(s) positioned at the tip 60 of the catheter 50, wherein the transmitter(s) or receiver(s) at the tip 60 of the catheter 50 are in operative communication with a corresponding electromagnetic transmitter and/or receiver disposed external to the patient's body. The electromagnetic catheter position guidance system may track the positioning and placement of the tip 60 of the catheter 50 in real-time, e.g., tracing a path of placement of the tip 60 or measuring a distance traversed within the patient's body. Thus, the electromagnetic catheter position guidance system may provide a complementary method for a user to determine when the tip 60 of the catheter 50 has passed the epiglottis 87 of the patient and indicate that the sensor(s) 46, 56 should begin to sense the carbon dioxide and/or pressure levels by sampling air from the lumen 70 of the catheter 50. The electromagnetic position guidance system may be used in conjunction with the markings 112, on its own, or in conjunction with any other suitable method for determining the depth of insertion of the tip 60 of the catheter 50 within the patient's anatomy.

Now that the specific components of the catheter guidance system 2 have been discussed in detail, a method of using the catheter guidance system 2 of the present invention in order to verify the accurate placement of a catheter 50 used for enteral feeding in the digestive tract is discussed in more detail below with reference to FIGS. 6A-7C.

Generally, the method for determining if the catheter 50 is accurately placed within a digestive tract of a body 78 of a patient includes inserting a distal end of the tubing assembly 14 (e.g., the distal end or tip 60 of the catheter 50) into an orifice 72 of the body 78, such as a nostril 87 of the patient's nose. As described above, the tubing assembly 14 can include the catheter 50 and at least one sensor, either in the form of the one or more sensor(s) 56 of the aspiration device 52 or the sensor 46 within the catheter 50. Once the tubing assembly 14 is inserted into the orifice 72 of the body 78, the sensors 46, 56 can be electrically connected to a processor 20 via a wired connection, although a wireless connection is also contemplated by the present invention such that no wire assembly or controller coupler is required.

In one aspect, the aspiration device 52 may deliver a positive pressure of air flow through the catheter 50 when the distal end 60 of the catheter 50 is inserted. The positive pressure of air may be delivered until the user determines that the distal end 60 of the catheter 50 has passed the epiglottis 90 of the patient. For instance, when the markings 112 are no longer visible to the user, the user may interpret that the distal end 60 of the catheter 50 has likely passed the epiglottis 90 of the patient. Notably, the epiglottis 90 is the point at which the respiratory tract, e.g., trachea 92, diverges from the digestive tract, e.g., esophagus 91. By delivering a positive pressure of air through the distal tip 60 of the catheter 50 until the distal tip 60 has passed the epiglottis 90, the likelihood that water, fluid, mucus or other substances that may be present within the patient's nostril 87 or nasopharynx 89 will be aspirated or sucked into the catheter 50 is significantly reduced. Moreover, the sensing by the sensor(s) 46, 56 need not be initiated until after the distal tip 60 passes the epiglottis 90, at which point the sensing can be used to differentiate between the positioning of the distal tip 60 in the digestive tract or the respiratory tract.

Next, the sensor(s) 46, 56 are activated, and the sensor(s) 46, 56 then begin to continuously measure the carbon dioxide concentration, the pressure and/or airflow or a combination thereof from air in the lumen 70 of the catheter. The aspiration device 52 may be switched to a vacuum suction or negative pressure mode to pull a small amount of air from the lumen 70. The vacuum suction or negative pressure can be continuous or intermittent, which may be important for preventing hypoxia in pediatric or neonatal patients. For instance, the aspiration device 52 may pull about 0.15 mL/sec to about 0.40 mL/sec of air from the lumen 70 of the catheter 50 in order to draw air past the sensors 56, e.g., carbon dioxide sensor 220, pressure sensor 230 and/or flow sensor 240. The sensors 220, 230, 240 communicate with the processor 260 of the aspiration device 52 to deliver carbon dioxide readings, pressure readings, flow readings, or a combination thereof to the processor 260 in real-time, and the processor 260 may be further coupled to communicate with the processor 20. In an embodiment in which a sensor 46 is present within the catheter 50, the sensor 46 communicates with the processor 20 via the wired connection (e.g., wire assembly 62) or the wireless connection to deliver carbon dioxide readings, pressure readings, flow readings, or a combination thereof to the processor 20 in real-time.

In addition, a display device 22 is coupled to the processor 20 and displays the carbon dioxide readings, pressure readings, flow readings or a combination thereof communicated by the sensor(s) 46, 56 for a health care provider to use during the catheter insertion procedure. For instance, as the distal end or tip 60 of the catheter 50 is advanced inside the body 78 in a direction away from the orifice 72 while the sensor(s) 46 and/or 56 are activated, the carbon dioxide readings, pressure readings, or a combination thereof are observed or monitored on the display device 22.

Specifically, a generally constant, low concentration carbon dioxide profile, a generally constant or decreasing negative pressure profile, or both a combination thereof displayed or otherwise communicated by the display device 22 after a pre-determined amount of time indicates placement of the catheter 50 in a digestive tract (e.g., esophagus 91, stomach 74, intestine 96, or other anatomical region of the digestive tract of a patient. On the other hand, a non-constant or variable (e.g., sinusoidal wave, square wave, etc.) carbon dioxide profile displayed or otherwise communicated by the display device 22 after a pre-determined amount of time indicates placement of the catheter 50 in the respiratory system (e.g., trachea 92, bronchi 93, lungs 94, or other anatomical region of the digestive tract of the patient). If the procedure is, e.g., insertion of a feeding tube intended for placement in the digestive tract, then at the time of detection of catheter placement in the respiratory tract the insertion procedure should be stopped immediately and the tubing assembly 14 be removed from the respiratory tract to avoid potential harm to the patient. Further, in order for such information to be displayed or otherwise communicated by the display device 22, a memory device 21 stores instructions which, when executed by the processor 20, cause the processor 20 to (i) interpret the carbon dioxide readings, the pressure readings, or a combination thereof communicated by the sensor(s) 46 and/or 56 and (ii) cause the display device 22 to communicate whether or not the catheter 50 is placed within the digestive tract of the patient based on the interpretation of the carbon dioxide readings, the pressure readings, or a combination thereof.

The present inventors have found that the distinctions between the carbon dioxide and/or pressure profiles of air sampled from the lumen 70 of the catheter, either via placement of the sensor(s) 56 in the aspiration device 52 upstream, where the air sampled is obtained from the lumen 70 via suction from a vacuum pump 58, or placement of the sensor 46 in the lumen 70 of the catheter 50 itself, when the distal end or tip 60 of the catheter 50 is placed within the digestive tract or respiratory system allow for an efficient and possibly life-saving determination of accurate enteral feeding catheter 50 placement in the digestive tract, where erroneously placing the catheter in the respiratory system would deliver fluid into the lungs or damage lung tissue, which can have fatal consequences.

The aspiration device 52 is configured to generate a low level of vacuum suction that is continuously pulled through the catheter 50. As the catheter 50 is advanced through the body, pressure readings detected by the sensor(s) 46 and/or 56 change based on the vacuum resistance (i.e., negative pressure) sensed at the distal end 60 of the catheter 50. For example, when the distal end 60 of the catheter 50 is in free airspace, such as the trachea, the vacuum (negative) pressure signal will be low. Whereas, if the distal end 60 of the catheter 50 is in contact with tissue, e.g. in the esophagus, the vacuum (negative) pressure signal will be higher. The display device 22 may provide information regarding the location of the distal end 60 of the catheter 50, such as in the form of a graph 37 (see FIGS. 6C and 7C). The y-axis of the graph 37 corresponds to vacuum pressure signal and the x-axis of the graph corresponds to time. Although, in other embodiments, the y-axis may correspond to time and the x-axis may correspond to vacuum pressure signal. (Not shown). Accordingly, the graph 37 may illustrate the vacuum pressure at the distal end 60 of the catheter 50 over time. As shown in FIG. 6C, when the distal end of the tube 60 is in the esophagus or gastrointestinal tract, the graph 37 will begin showing areas of higher vacuum pressure as compared to the baseline vacuum pressure. As shown in FIG. 7C, however, when the distal end of the tube 60 is in the trachea or respiratory tract, the graph 37 will begin showing areas of lower vacuum (negative) pressure as compared to the baseline vacuum pressure. Accordingly, differentiating between these two signals allows for location identification of the distal end 60 of the catheter 50 to be known in real time throughout the course of placing the catheter 50 in the patient's body. Thus, the location of the distal end 60 of the catheter 50 can be made as follows: (1) if the sensor 46 and/or 56 begins to measure a higher vacuum resistance (more negative pressure) within the catheter 50, then the distal end 60 of the catheter 50 is in the esophagus 91 and placement can continue through the digestive tract, but (2) if the sensor 46 and/or 56 measures no change in the vacuum resistance of the catheter 50 or a lower vacuum resistance (higher pressure) within the catheter 50, the distal end 60 of the catheter 50 is in the airway, e.g. the trachea 92 or lungs 94, and the catheter 50 should be repositioned.

Moreover, the aspiration device 52 can be implemented to confirm whether a detected vacuum resistance within the catheter 50 is due to placement of the catheter 50 within the esophagus 91 or due to occlusion (e.g., debris such as food particulate, mucus, fluid, etc.) of the distal tip 60 of the catheter 50. For instance, the aspiration device 52 can be used to deliver one or more “puffs” or bursts of positive air pressure followed by immediately resuming suction through the catheter 50. If vacuum resistance is immediately obtained following the puff or burst of positive air pressure, then the user can infer that the catheter 50 is placed within the esophagus 91. However, if vacuum resistance is not immediately obtained following a puff or burst of positive air pressure, then the catheter 50 may be in the airway and the sensor 46 may continue to look for airway signals such as elevated carbon dioxide levels and/or free flow of air through the catheter 50.

Additionally, a flow sensor can be incorporated into the sensor(s) 46 and/or 56. A free flow of air within the catheter 50 may indicate placement of the catheter 50 within the airway of a patient, particularly when coupled with an elevated level of carbon dioxide.

For instance, as shown in FIGS. 6A, 6B, and 6C, when the distal end or tip 60 of the catheter 50 is inserted into the nostril 87 of the patient and is advanced through the nasal cavity 88, past the nasopharynx 89, and into the esophagus 91 just past the epiglottis 90, as the sensor(s) 46 and/or 56 are continuously sampling air from the lumen of the catheter 50 over time in seconds, the carbon dioxide level (FIG. 6B) and pressure (FIG. 6C) graphs displayed or otherwise communicated by the processor 20, such as via the display device 22, may initially show non-constant readings, but ultimately reach a generally constant or decreasing level over time as the distal end or tip 60 of the catheter 50 travels into the digestive tract and not into the respiratory system. With insertion of the catheter 50 accurately into the digestive tract, the generally constant readings are ultimately obtained within a matter of seconds of the insertion procedure once the distal end or tip 60 reaches the esophagus 91 and is not exposed to the pattern of breathing associated with inspiration and expiration, where the carbon dioxide and pressure levels rise and fall in a repetitive pattern.

On the other hand, as shown in FIGS. 7A, 7B, and 7C, when the distal end or tip 60 of the catheter 50 is inserted into the nostril 87 of the patient and is advanced through the nasal cavity 88, past the nasopharynx 89, and into the trachea 92 just past the epiglottis 90, and then into the bronchi 93 or lungs 94, as the sensor(s) 46 and/or 56 are continuously sampling air from the lumen of the catheter 50 over time in seconds, the carbon dioxide level (FIG. 7B) and pressure (FIG. 7C) graphs displayed or otherwise communicated by the processor 20, such as via the display device 22, show non-constant readings over time as the distal end or tip 60 of the catheter 50 travels into the respiratory system. With insertion of the catheter 50 inaccurately into the respiratory system, constant carbon dioxide and pressure readings are not obtained due to the pattern of breathing associated with inspiration and expiration. This will ultimately be apparent to the health care provider within a matter of seconds of the insertion procedure once the distal end or tip 60 reaches the trachea 92, the bronchi 93, or the lungs 94, as the distal end or tip 60 of the catheter will be exposed to the pattern of breathing associated with inspiration and expiration, where the carbon dioxide and pressure levels rise and fall in a repetitive pattern, have a higher baseline level than the esophagus, and do not reach constant levels. At this point, the health care provider can be alerted to remove the tubing assembly 14 from the respiratory system and start a new procedure to accurately place the distal end or tip 60 of the catheter 50 into the digestive tract for enteral feeding.

Further, as an alternative or in addition to monitoring the carbon dioxide and/or pressure readings as determined by the sensor(s) 46 and/or 56 over time and observing the change from non-constant or oscillating readings to constant readings, the health care provider can also verify accurate placement of the catheter 50 in the esophagus 91 rather than the trachea 92 by observing for the presence or absence of a plurality of markings 112 uniformly spaced along the external surface of the catheter. As described above, such markings 112 can be used in conjunction with the sensor 46 to determine accurate placement of the catheter 50. These markings 112 can function as placement markers which assist the user in assessing the depth that the catheter 50 is placed within the body 78. For instance, the markings 112 can be present from the distal end 60 of the catheter 50 to a point 126 on the catheter 50 that spans a distance that can correspond with the average distance between the trachea 92 and nostril 87 in a typical patient. As the catheter 50 is being inserted into the body 78 via the nostril 87, once the markings 112 are no longer visible outside the body 78, the health care provider can initiate sensing of the carbon dioxide and/or pressure levels. If the carbon dioxide and/or pressure readings are still oscillating to the analog of breathing once the markings 112 are no longer visible outside the body 78, then the health care provider will know that the catheter 50 has been improperly inserted into the trachea 92 instead of the esophagus 91, and the catheter 50 can be immediately retracted.

Notably, the catheter guidance system 2 of the present invention may be further used to guide and determine the correct placement of an enteral feeding tube even when a patient is intubated with an endotracheal tube for mechanical ventilation. In such instances, the sensor(s) 46 and/or 56 may not be activated until the distal tip 60 of the catheter 50 has extended a distance into the patient's body that is determined to be roughly equal to or longer than the distance from the nostril 87 to the trachea 92, as there would be little to no breathing pattern of inspiration or expiration above the point at which a cuff of the endotracheal tube is placed within the trachea 92.

Regardless of the particular method by which proper placement of the catheter 50 is determined, once the distal end or tip 60 of the catheter 50 has been accurately placed within the desired location in the digestive tract, the health care provider can then optionally remove or disconnect the sensor 46, while the position of the catheter 50 is maintained. The health care provider can then attach medicine and nutritional delivery tubes to the y-port connector 44 for introducing fluids into the body (e.g., digestive tract) for medical treatment. On the other hand, if the sensor 46 is wireless, the sensor 46 can optionally be left in place, and the health care provider can then attach medicine and nutritional delivery tubes to the y-port connector 44 for introducing fluids into the body (e.g., digestive tract) for medical treatment.

It should also be appreciated that the tubing assembly, electronic catheter unit and catheter position guidance system of the present invention can be used in a variety of catheter procedures and applications. These procedures may involve the treatment of the digestive or gastrointestinal tract or other portions of the human body. Additionally, these procedures may involve the treatment of the respiratory tract, such as the correct positioning of an endotracheal tube. These procedures may involve treatment of humans by physicians, physician assistants, nurses or other health care providers. In addition, these procedures may involve treatment of other mammals and animals by veterinarians, researchers and others.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

1-25. (canceled)

26. A catheter sensor assembly comprising:
a catheter having a proximal end and a distal end and extending in a longitudinal direction, wherein the proximal end and the distal end define a lumen therebetween, and wherein the catheter is configured for placement within a digestive tract or airway of a patient;
an aspiration device removably coupled to the proximal end of the catheter, wherein when the distal end of the catheter is being advanced into the patient, the aspiration device delivers positive air pressure through the lumen of the catheter to the distal end of the catheter until the catheter has reached a predetermined anatomical location;
a plurality of sensors located within the aspiration device and configured to continuously obtain real-time measurements from the lumen independently of the patient's inspiration and expiration and subsequent to the catheter passing the patient's epiglottis, wherein the plurality of sensors comprises a carbon dioxide sensor and a pressure sensor; and
a processor in electronic communication with the plurality of sensors configured to determine whether the distal end of the catheter is located within the digestive tract of the patient based, at least in part, on a profile of the real-time measurements corresponding with a position of the catheter in a particular anatomical location or region,
wherein a low concentration carbon dioxide profile and a constant or decreasing pressure profile indicate placement in the digestive tract and a variable carbon dioxide profile and a negative relative pressure indicate placement in the airway.

27. The catheter sensor assembly of claim 26, wherein at least one sensor is located at the distal end of the catheter.

28. The catheter sensor assembly of claim 26, wherein the plurality of sensors is configured for a wired connection or a wireless connection to the processor.

29. The catheter sensor assembly of claim 26, wherein the aspiration device is configured to draw a small volume of air from the lumen of the catheter.

30. The catheter sensor assembly of claim 26, wherein the plurality of sensors comprises a flow rate sensor, and wherein the processor is configured to determine whether the distal end of the catheter is located within the digestive tract of the patient based, at least in part, on flow rate information obtained via the flow rate sensor.

31. The catheter sensor assembly of claim 26, wherein the delivery of positive pressure of air to the distal end of the catheter is configured to differentiate between placement of the distal end of the catheter in the esophagus and occlusion of the distal end of the catheter when the distal end of the catheter is placed in the airway.

32. A catheter guidance system comprising:
(a) a processor;
(b) a power source;
(c) a display device; and
(d) a catheter sensor assembly comprising:
a catheter having a proximal end and a distal end and extending in a longitudinal direction, wherein the proximal end and the distal end define a lumen therebetween;
an aspiration device removably coupled to the proximal end of the catheter, wherein when the distal end of the catheter is being advanced into a patient, the aspiration device delivers positive air pressure through the lumen of the catheter to the distal end of the catheter until the catheter has reached a predetermined anatomical location; and
a plurality of sensors located within the aspiration device and configured to continuously obtain real-time measurements from the lumen independently of the patient's inspiration and expiration and subsequent to the catheter passing the patient's epiglottis, wherein the plurality of sensors comprises a carbon dioxide sensor and a pressure sensor,
wherein the plurality of sensors communicates with the processor via an electrical connection to deliver carbon dioxide readings and pressure readings corresponding with a position of the catheter in an anatomical location or region as measured by the plurality of sensors from air in the lumen in real-time;
wherein the display device is coupled to the processor and displays the carbon dioxide readings and pressure readings communicated by the plurality of sensors;
wherein the processor is configured to determine whether the distal end of the catheter is located within the patient's digestive tract based, at least in part, on a carbon dioxide reading profile and a pressure profile after a pre-determined amount of time as shown on the display device to indicate placement of the catheter in a digestive tract or an airway of the patient, and wherein a low concentration carbon dioxide profile and a constant or decreasing pressure profile indicate placement in the digestive tract and a variable carbon dioxide profile and a negative relative pressure indicate placement in the airway.

33. The catheter guidance system of claim 32, wherein the catheter guidance system further includes at least one navigational guide configured to indicate when the distal end of the catheter has passed the epiglottis of the patient when the distal end of the catheter is inserted through the patient's nose or mouth.

34. The catheter guidance system of claim 32, further comprising a memory device storing instructions which, when executed by the processor, cause the processor to
cause the display device to communicate whether the catheter is placed within the digestive tract of the patient or the airway of the patient based on the interpretation of the carbon dioxide readings and the pressure readings.

35. The catheter guidance system of claim 32, wherein the plurality of sensors comprises a flow rate sensor, and wherein the processor is configured to determine whether the distal end of the catheter is located within the digestive tract of the patient based, at least in part, on flow rate information obtained via the flow rate sensor.

36. A method for determining if a catheter is placed within a digestive tract or an airway of a body of a patient, the method comprising steps of:
(a) inserting a distal end of a tubing assembly into an orifice of the body, wherein the tubing assembly comprises:
the catheter, wherein the catheter has a proximal end and a distal end and extends in a longitudinal direction, wherein the proximal end and the distal end define a lumen therebetween;
an aspiration device removably coupled to the proximal end of the catheter, wherein when the distal end of the catheter is being advanced into the patient, the aspiration device delivers positive air pressure through the lumen of the catheter to the distal end of the catheter until the catheter has reached a predetermined anatomical location and
a plurality of sensors located within the aspiration device and configured to continuously obtain real-time measurements from the lumen independently of the patient's inspiration and expiration and subsequent to the catheter passing the patient's epiglottis, wherein the plurality of sensors comprises a carbon dioxide sensor and a pressure sensor
(b) activating the plurality of sensors, wherein the plurality of sensors measures carbon dioxide and pressure from air in the lumen and communicates with a processor via a wired connection or a wireless connection to deliver carbon dioxide readings, pressure readings, or a combination thereof corresponding with a position of the catheter in an anatomical location or region to the processor in real-time, wherein a display device is coupled to the processor and displays the carbon dioxide readings and pressure readings communicated by the plurality of sensors;
(c) advancing the distal end of the catheter inside the body in a direction away from the orifice while the plurality of sensors is activated subsequent to passing the patient's epiglottis, and
(d) observing the carbon dioxide readings and pressure readings on the display device, wherein a carbon dioxide reading profile and a pressure reading profile, after a pre-determined amount of time indicates placement of the catheter in a digestive tract or an airway of a patient, wherein a low concentration carbon dioxide profile and a constant or decreasing pressure profile indicate placement in the digestive tract and a variable carbon dioxide profile and a negative relative pressure indicate placement in the airway.

37. The method of claim 36, wherein the orifice is a nose or a mouth.

38. The method of claim 36, wherein suction from the aspiration device directs air sampled from a distal end of the catheter to the plurality of sensors.

39. The method of claim 38, further including a step of delivering a positive pressure of air from the aspiration device through the distal end of the catheter while inserting the distal end of the catheter inside the body in a direction away from the orifice until the distal end of the catheter reaches a predetermined anatomical reference point.

40. The method of claim 39, wherein steps (b) and (c) are performed after the distal end of the catheter reaches the predetermined anatomical reference point.

41. The method of claim 36, further comprising a step of: providing at least one navigational guide, wherein information from the at least one navigational guide is configured to indicate placement of the catheter in the digestive tract or airway of the patient.

42. The method of claim 36, wherein a memory device stores instructions which, when executed by the processor, cause the processor to
cause the display device to communicate whether or not the catheter is placed within the digestive tract of the patient based on the interpretation of the carbon dioxide readings and the pressure readings.

43. The method of claim 36, wherein the plurality of sensors comprises a flow rate sensor, and wherein the method includes:
determining whether the distal end of the catheter is located within the digestive tract of the patient based, at least in part, on flow rate information obtained via the flow rate sensor.
